(19) United States
(12) Patent Application Publication
(10) Pub. No.: US 2007/0258910 A1
(43) Pub. Date: Nov. 8, 2007

(54) Methods and devices for remineralization of hard tissues

(76) Inventor: Dwayne D. Arola, Baltimore, MD (US)

Correspondence Address: Benjamin Aaron Adler;ADLER & ASSOCIATES, 8011 Candle Lane, Houston, TX, 77071, US

(21) Appl. No.: 11/800,850

(22) Filed: May 8, 2007

Related U.S. Application Data
(60) Provisional application No. 60/798,701, filed on May 8, 2006.

(51) Int. Cl. A61K 33/42; A61K 9/14

(52) U.S. Cl. 424/46; 424/603

(57) ABSTRACT

Provided herein are methods to remineralize the demineralized areas of hard tissues. The disclosure provides for remineralizing methods and systems that utilize a jet stream laden with particles comprising at least calcium and phosphorus to remove demineralized zone and implant particles within the hard tissue to stimulate remineralization. Some aspects of the disclosure can be advantageously used to remineralize one or more of enamel, dentin, and bone.

### CROSS-REFERENCE TO RELATED APPLICATION

This nonprovisional application claims benefit of provisional U.S. Ser. No. 60/798,701, filed May 8, 2006, now abandoned.

### BACKGROUND OF THE INVENTION

1. Field of the Invention

The present invention relates to the fields of preventative dentistry and medical treatments effective to promote remineralization of hard tissues. More specifically, the present invention relates to methods and systems for facilitating remineralization of hard tissues by depositing particles into the demineralized areas of the hard tissues using a jet device.

2. Description of the Related Art

Demineralization and remineralization are dynamic processes that have a vital impact on hard tissues. Demineralization is the process of removing minerals in the form of mineral ions, for example, from dental enamel or bone. Several factors contribute to the formation of demineralized zones. Demineralization of enamel often is caused by acids found in acidic foods, such as tomatoes or oranges, or is generated by oral bacteria that feed on starches and sugars in the mouth, especially refined sugars, and secrete acids as by-products. Remineralization is the process of restoring minerals, as mineral ions, to the hydroxyapatite latticework structure.

Demineralized hard tissue is currently managed by preventive treatment, the removal of demineralized zones and/or the protection of any newly exposed non-carious dentine with restorative material. Measures aimed at the prevention or the arrest of dental caries are based primarily on the elimination of dental plaque from the surfaces of roots and the institution of dietary controls to reduce the frequency and quantity of readily fermentable carbohydrate ingestion.

Conventional removal of demineralized zones is used often in dentistry to eradicate decayed dentin and entails the use of high and low speed handpieces. However, the perception that drilling is unpleasant for patients and that local anesthetic is frequently required are disadvantages of this system. In addition, handpieces are expensive to purchase and maintain and their use may lead to the removal of softened, but uninfected, dentine resulting in the excessive loss of tooth tissue. Where restoration is required, all materials used to restore demineralized zones have their limitations. For example, gold and ceramic are expensive and present a technical challenge for the practitioner. While amalgam is a durable, predictable material, it has poor aesthetic qualities, is potentially toxic and may cause allergic reactions in some people.

Though caries may be identified at their incipient stages, for example, using techniques such as laser fluorescence detection (LFD), the currently available techniques for restoring demineralized zones are limited. If demineralization is at its early stages and has not propagated into the dentin, the structural integrity of the enamel may not be significantly decreased. In these cases remineralization of the enamel can be the most non-invasive technique for restoration. However, the currently available methods of remineralization are not fully satisfactory in that the needs described above are not met. In some instances, some of the available methods may not effectively provide sufficient minerals in the proximity of carious activity to remineralize the enamel.

Thus, there is a recognized need in the art for improved methods and systems for remineralization of hard tissues. Specifically, the prior art is deficient in methods and systems that utilized air jet devices to deliver particles capable of remineralizing hard tissues in an individual. The present invention fulfils this longstanding need in the art.

### SUMMARY OF THE INVENTION

The following presents a simplified summary in order to provide a basic understanding. This summary is not intended to identify key or critical elements nor to delineate scope. A purpose of this summary is to present some concepts in a simplified form as a prelude to the more detailed description later presented. Additionally, section headings used herein are provided merely for convenience and should not be takes as limiting in any way.

Generally, the subject invention discloses methods to remineralize the demineralized areas of hard tissues. Specifically, the present invention discloses remineralization methods that utilize a jet delivery device adapted to deliver particles comprising at least calcium and phosphorus to remove the demineralized zone and impregnate particles within the hard tissue to stimulate remineralization. The hard tissues can be one of dentin, enamel, or bone.

One object of the present invention is to provide a method for remineralizing the deminerailized areas of selected hard tissues. Another object of the present invention is to provide a method for remineralizing localized zones of dental enamel or dentin that have undergone demineralization as a result of bacterial invasion.

Yet another object of the present invention is to provide a method for delivering particles to the demineralized zone of a hard tissue such that the particles remove the demineralized region and embed particles comprising at least one or more of calcium or phosphorus into the hard tissue using an air jet device.

Thus, the present invention is directed to a method for remineralizing a hard tissue in a subject. The method comprises directing a pressurized stream of particles comprising one or both of calcium or phosphorus onto a site of interest on the hard tissue thereby implanting at least a portion of the particles therewithin. The implanted particles are effective to deliver one or both of calcium or phosphorus ions to the hard tissue thereby reminerlizing the hard tissue in the subject. The present invention is directed to a related method of remineralizing hard tissue comprising a further method step of positioning a nozzle operably attached to a jet delivery device adapted to deliver the particles therethrough proximately to the site of interest on the hard tissue.

The present invention also is directed to a method for remineralizing a tooth in a subject. The method comprises delivering, via a jet device, a plurality of apatite or hydroxyapatite particles to a site of demineralization on the tooth at a flow rate whereby at least a portion of the apatite particles are implanted therein. The implanted apatite or hydroxyapatite particles are effective to deliver both calcium and phosphous ions to the demineralized site thereby remineralizing the tooth in the subject. In a related tooth remineralization method the apatite or hydroxyapatite particles further may comprise one or more abrasive particles.

The present invention is directed further to a method for remineralizing a bone in a subject. The method comprises delivering, via a jet device, a plurality of particles comprising one or both of calcium or phosphorus to a site of demineralization on the bone at a flow rate whereby at least a portion of the particles are implanted therein. The implanted particles are effective to deliver one or both of calcium or phosphous ions to the demineralized site thereby remineralizing the bone in the subject. In a related bone remineralization method the particles may be or may include one or more abrasive particles.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 depicts a simplified diagram of an air jet device for use in remineralization of hard tissues according to the present invention.

FIG. 2 depicts a more detailed schematic of an air jet device for remineralizing of hard tissues according to the present invention.

FIG. 3 is a flowchart depicting method steps for remineralizing of hard tissues according to the present invention.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, the term “subject” refers to any recipient of hard tissue remineralizatin as described herein.

In one embodiment of the present invention there is provided a method for remineralizing a hard tissue in a subject, comprising directing a pressurized stream of particles comprising one or both of calcium or phosphorus onto a site of interest on the hard tissue thereby implanting at least a portion of the particles therewithin, where the implanted particles are effective to deliver one or both of calcium or phosphorus ions to the hard tissue thereby reminerlizing the hard tissue in the subject.

Further to this embodiment, the method for remineralizing may comprise positioning a nozzle operably attached to a jet delivery device adapted to deliver the particles therethrough proximately to the site of interest on the hard tissue. In this further embodiment, the nozzle may have a diameter of about 0.005 mm to about 10 mm. Also, the nozzle may be positioned about 1 mm to about 25 mm from the hard tissue. In addition, the nozzle may be angled or may be positioned at an angle from the hard tissue. Particularly, the nozzle may be positioned at an angle of about 0 degrees to about 180 degrees relative to the hard tissues. Furthermore, the device to which the nozzle is fluidly attached may be an air jet device or an aqueous jet device.

In both embodiments, a component mass of a particle is about 2 percent to about 30 percent phosphorus and about 25 percent to about 60 percent calcium. Particularly, the particles may be apatite or hydroxyapatite. Also, the particles may further comprise one or more abrasives. Representative examples of abrasives may include aluminum oxide, crushed glass, glass beads, plastic media, silicon carbide, sodium bicarbonate, or walnut shell. In addition particle size may be about 0.001 μm to about 1000 μm and particles may have an absolute hardness of about 0.1 to about 2000.

Also, in both embodiments the pressurized stream may be an air stream or an aqueous stream. In addition, the particles may be deposited onto the hard tissue at a flow rate of about 0.1 g/s to about 5.0 g/s. Particularly, the hard tissue may be enamel, dentin or bone. Furthermore, the site of interest may be a bacterial lesion or a demineralized area of the hard tissue.

In another embodiment of the present invention, there is provided a method for remineralizing a tooth in a subject, comprising delivering via a jet device a plurality of apatite particles to a site of demineralization on the tooth at a flow rate whereby at least a portion of the apatite particles are implanted therein; where the implanted apatite particles are effective to deliver both calcium and phosphous ions to the demineralized site thereby remineralizing the tooth in the subject.

In this embodiment, the apatite particle size may be about 1 μm to about 1000 μm. Particularly, the apatite may be a hydroxyapatite with a particle size of about 1 μm to about 100 μm. Also, the apatite particles further may comprise one or more abrasives including aluminum oxide, crushed glass, glass beads, plastic media, silicon carbide, sodium bicarbonate, or walnut shell. In addition, the apatite particles may be delivered to the site of demineralization at an angle of about 0 degrees to about 180 degrees relative to the tooth. Furthermore, the particles have a hardness of about 1 Mohs to about 20 Mohs. The site of demineralization may be one or both of the enamel or dentin. The particle mass composition, the particle absolute hardness and the flow rate are as described supra.

In yet another embodiment of the present invention, there is provided a method for remineralizing a bone in a subject, comprising delivering via a jet device a plurality of particles comprising one or both of calcium or phosphorus to a site of demineralization on the bone at a flow rate whereby at least a portion of the particles are implanted therein; where the implanted particles are effective to deliver one or both of calcium or phosphous ions to the demineralized site thereby remineralizing the bone in the subject. In this embodiment the particle mass composition, the particle size and hardness, the abrasives further comprising the particles, the angle of particle delivery, and the flow rate are as described supra.

The present invention discloses methods that facilitate remineralization of hard tissues. In particular, the present invention provides for the remineralization of demineralized areas of the hard tissues using air or aqueous jet devices that deliver particles containing calcium and phosphorus, such as, but not limited to different types of apatite. One or more embodiments of the present invention can be advantageously used to remineralize the hard tissues such as enamel, bone and dentin. The methods and jet devices of the present invention may be used to remove a bacterial lesion and/or the demineralized zone of a hard tissue and impregnate the particles within the hard tissues to stimulate remineralization.

Remineralization using the methods and jet devices described herein can serve as a non-invasive technique for restoration of the structural integrity of hard tissues such as enamel at early stages of demineralization when demineralization has not yet propagated into the dentin. For example, due to the softening of enamel from demineralization, the particles of the present invention can be implanted with higher density within regions of enamel that have undergone the demineralization. One or more methods of the present invention have the potential of being advantageous over existing remineralizing gums that may not provide sufficient minerals in proximity to carious activity or may not allow sufficient time for mineral ions to diffuse within the region of affected enamel.

The claimed subject matter is now described with reference to the drawings, wherein like reference numerals are used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, well-known structures and devices are shown in block diagram form in order to facilitate describing the claimed subject matter.

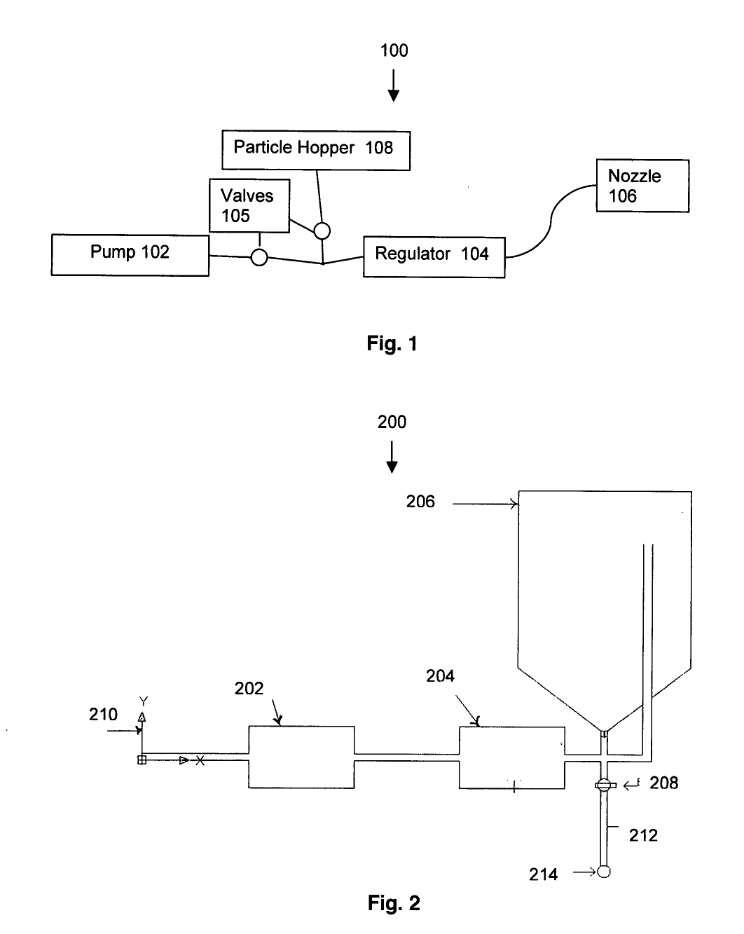

FIG. 1 is a simplified schematic of an air jet device 100 useful in the methods for remineralizing the demineralized areas of hard tissues provided herein. Many jet devices are commercially available as complete systems with many components required for pressure generation or intensification, such as cabinets, nozzles or wheels, and hoppers and collectors, or they may be purchased in component form to either build a custom system for this invention. Abrasive jet devices includes four main types of devices (not including proprietary or custom devices, which can be designed by suppliers as necessary), abrasive water jet devices, air abrasive jet devices, and precision jets or micro-jets. Abrasive water jet devices use abrasive particles entrained in a stream of water to deposit the particles. Air abrasive jet devices are employed in similar circumstances, although they use air as the propelling fluid. Air abrasive systems are used in various industries. While the device and method will be described primarily in terms that are suitable for dental applications, it is to be understood that the device and method can be modified to be used in other hard tissues, such as bone.

Thus, generally, the air jet device 100 may be any jet device capable of generating pressurized stream laden with particles against a surface. Any commercially available jet devices, such as air jet devices or abrasive air jet devices or aqueous jet devices may be used for depositing or delivering the particles containing calcium and/or phosphorous into the hard tissues with or without modification. Jet devices or abrasive jet devices may be used to deposit particles by generating a focused stream of fluid, e.g., water, or compressed air or other gas mixed with particles. They may use pressurized water or compressed air or gas as the driving fluid to propel the particles.

More particularly, the air jet device 100 includes at least one pump module 102 capable of developing sufficient air pressure to deposit particles comprising one or both of calcium and phosphorus, a regulator module 104 includes one or more of valves, meters, etc. 105 that are capable of controlling mass flow rate of air and the particles, a nozzle module 106 that can be used in depositing the particles into the demineralized areas, and a particle holding module 108 for holding the particles. The pump module 102, the regulator module 104, the nozzle module 106, and the particle holding module 108 are operably and fluidly coupled to form the jet device 100 that can facilitate the delivery of the particles to the hard tissues.

The pump module 102 may be any pump system that can generate sufficient pressure useful to deliver the particles. Many pump systems are known in the art and a person skilled in the art would appreciate that they can be used to deliver the particles of this invention with or without modification. The regulator module 104 can be any system that can regulate or control the flow rate, the pressure, the velocity of the jet stream laden particles. The regulator can be any plumbing system known in the art of jet devices. Typically, the regulator module 104 includes valves, meters, mixers and others which are included at 105.

The nozzle module 106 of device 100 can be any known in the art. The nozzle module 106 may have additional components besides nozzle such as hand pieces, flow rate regulators, flow directors, etc. A more detailed description of nozzle properties is provided infra.

The particle holding module 108 can be any system that can hold, maintain or supply the particles. In one example, the module 108 can be a hopper. In another example, the module can have other accessories attached to it such as mixers, meters, flow regulators, and etc.

FIG. 2 is a more detailed schematic of an air jet device 200 that produces a pressurized stream of gas and suspended abrasive particles useful in the methods described herein. Some examples of air jet devices are available in the market. Examples of the air jet device may include, but are not limited to the three KCP series (KCP 1000 Whisperjet, KCP 2000 and KCP 2000 Plus; American Dental Technologies), the Microprep (Sunrise Technologies), and the Kreativ (Kreative, Inc.). Optional accessory components for the device may include a mixing means for combining the pressurized gas with said abrasive particles, a means to produce the pressurized stream of gas and suspended particles, an inlet line for the pressurized gas to flow to the mixing means, or a mixing chamber positioned in the outlet line.

The air jet device 200 includes, a variable pressure regulator and gage means for controlling the pressure of the gas supplied 202, a pressure transducer means for conducting an electrical signal which causes oscillation of pressure to create a transient increase in velocity below the source of the particles 204, a source of particles 206, a valve means 208 for regulating the flow of pressurized gas an suspended particles to the nozzle, a source of pressurized gas 210, an outlet line 212 to carry the pressurized stream of gas and suspended particles, a nozzle 214 for delivering the pressurized stream of gas and suspended particles.

The air jet 200 may contain a pressure meter used to indicate the pressure in the hopper 206. The air jet may also utilize at least one hopper. At least one hopper is provided for selectively introducing apatite abrasive material into the gas flow stream. The hopper may consist of a closed cylinder with an air pressure supply line and an orifice in the bottom. The hoppers may be selectively connected by feed valves to a mixing chamber.

In one example, the air jet 200 utilizes a pressurized gas or air source having its output connected as an input to an electrically actuated valve. Although any compressed gas can be employed in the compressed gas source, compressed air is used in one embodiment. The gas may be any suitable inert, dry gas that is used in the air abrasive industry such as carbon dioxide, nitrogen, air, and the like. A person skilled in the art would appreciate that similar devices equipped with a source of compressed air may be available in many facilities which provide dental services. These devices may have a capacity to generate air pressures from about 10 psi to about and 500 psi. In some examples of these devices the air pressure can be from about 30 psi to about 70 psi.

The nozzle 214 may be conically tapered and pointed to reduce clogging of the nozzle. Nozzle shapes other than conical may be used. The nozzle 214 may have a diameter between 0.25 mm and 1.2 mm. The nozzle size along with the particle size may help determine where, if, and how the particle flow becomes turbulent. The nozzle may also be angled orthogonal to the surface of the hard tissue. The orthogonal angle allows for sufficient transfer of kinetic energy between the particles and surface of the hard tissue. This angle may also allow for consistency in shape cut in the enamel by the air jet. The kinetic energy produced aids in material removal and particle embedding. The air jet may also have an on/off valve which may be located on the nozzle line.

In one example, the air jet system allows the user to dilute the gas particle mixture for delivery to the surface. As dry air and abrasive powder or particles pass through the nozzle, they are channeled into a concentrated pattern that allows for enhanced cutting abilities and precision. The abrasive particles are of size and hardness ranges, flowability, shapes, and other characteristics that are appropriate for the purposes for which the air abrasive stream is to be used.

Although the above description is for air jet devices, the methods of the present invention may be used with fluid or aqueous jet devices. The use of a fluid jet, for example, but not limited to a water jet, with appropriate chemistry may facilitate increased rates of remineralization and/or increase the momentum transfer between the fluid medium and the hard tissues.

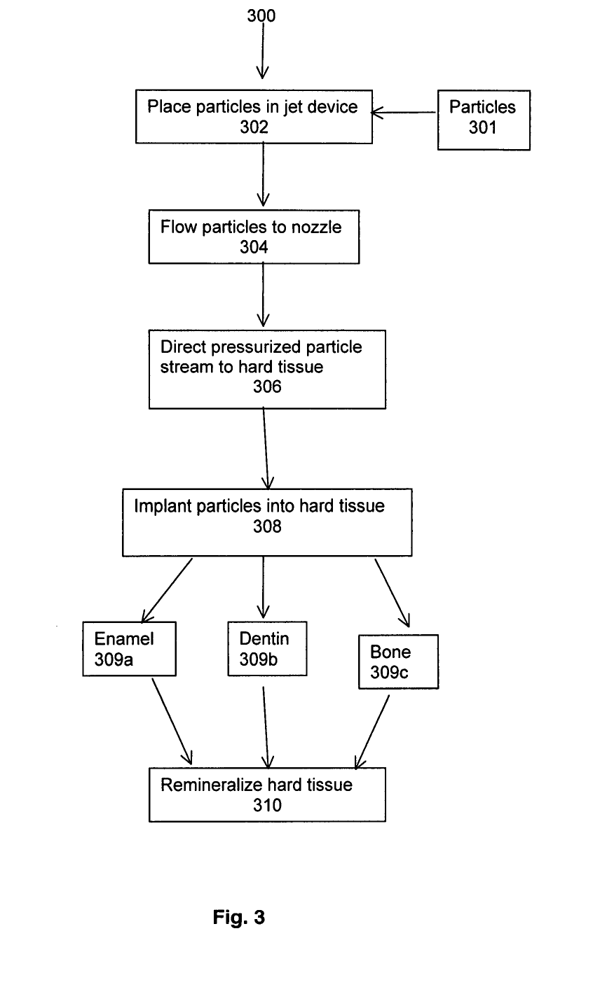

With reference to FIGS. 1-2, FIG. 3 depicts a method for remineralizing a hard tissue in a subject. The method 300 comprises a step 302 of placing a plurality of particles 301 in a jet device, for example, the simplified jet device 100 or more detailed jet device 200. At step 304 the particles are flowed under pressure to a nozzle, e.g. nozzle 106 or 214. At step 306 the pressurized stream of particles is directed onto areas of demineralization in a hard tissue. Subsequently, at step 308, the particles are implanted into the hard tissue, for example, enamel 309a, dentin 309b or bone 309c whereupon remineralization of the hard tissue occurs at step 310. It is contemplated that steps 306, 308 and 310 may be repeated as necessary.

The methods encompassed by general method 300 of the present invention may be used to remineralize hard tissues that are at an early stage of demineralization and have not yet propagated into the hard tissues. For example, early stages of demineralization can be identified with several emerging and established techniques such as laser fluorescence detection. Particularly, the method 300 may be used to remineralize tooth enamel 309a or dentin 309b exhibiting demineralized areas caused by, for example, but not limited to, bacterial lesions from carious activity. Alternatively, the method 300 may be used to enrich the mineral content of bone within regions that have been traumatized as a result of surgery or other pathophysiological conditions.

The particles 301 are chosen to have a) a sufficient size and shape for conforming to the jet device, b) sufficient hardness to allow removal of demineralized tissue and embedding in remaining tissue, and c) sufficient mass of calcium and phosphorous, to complement the hard tissue. The particles 301 useful in method 300 may be any particles that contain calcium and/or phosphorus. Also, the particles may have compositions similar to hard tissues. Non-limiting examples of the particles are monocalcium phosphate monohydrate, dicalcium phosphate anhydrous, tetra calcium phosphate, alpha tricalcium phosphate, calcium carbonate, beta tricalcium phosphate, hydroxyapatite, carbonated hydroxyapatite, calcium deficient hydroxyapatite, poorly crystalline hydroxyapatite, dicalcium phosphate dihydrate, amorphous calcium phosphate, calcium hydroxide, calcium fluoride, carbonate apatite thereof. Particularly, the particles may be apatite or hydroxyapatite. For example, hydroxyapatite has the well-known chemical formula Ca₅ (PO₄)₃OH.

The particles of the present invention may be formulated as a powder or may be granular. The particles may be crystalline, micro-crystalline, amorphous or any other shape. The shape of the particles may vary depending up on the application. For example, the shape of the particles may be spherical, angular, or any other contour that are known to persons skilled in the art. The particles also may be of various shapes, including, but not limited to, blocky shaped, blocky and sharp shaped, monoclinic shaped, and spherical shaped. For example, blocky shaped particles with points and edges are useful to cut and strip away surface material on impact.

The particles may act as an abrasive. Alternatively, the particles, in addition to comprising one or more of calcium and phosphorus also may include at least one of another abrasive, such as, but not limited to, aluminum oxide, crushed glass, glass beads, plastic media, Silicon carbide, sodium bicarbonate, or walnut shell.

The particles may have any suitable mass as long as they are suitable to sufficiently promote remineralization. In one example, the component mass of the particles may be about 2% to about 30% phosphorus and a mass of about 25% to about 60% calcium. In another example, the particles may have a component mass similar to hard tissues such as dentin, enamel, bone and cementum.

The particles may be of any suitable size. The particles may be mico-sized or nano-sized particles. In some examples the particles may be nano-apatite particles. Many of the particles for the present invention can be obtained commercially or can be made using the techniques known the art. For example, the size of the particles may range from about 0.001 micrometers (μm) to about 1000 μm. In another example, the size of the particles may range from about 0.01 μm to about 100 μm. In yet another example the size of the particles may range from about 1 micron to about 1000 μm. In yet another example, the size of the particles may range from about 10 μm to about 100 μm. As can be evident from the above, the size of the particles can vary as long as they are capable of facilitating remineralization. The particle size can also vary according to the application for which hey are being used.

The particles have a hardness sufficient to facilitate remineralization of demineralized areas of the chosen hard tissue. In one example, the particles have an absolute hardness ranging from about 0.1 to about 2000. In another example, the particles have a hardness ranging from about 1 Mohs to about 20 Mohs. More particularly, the particle hardness may be similar to enamel at 5.5 to 7 Mohs or dentin at 3.5-5.0 Mohs. The particles may be directed as a pressurize stream toward the hard tissues using a jet delivery device such that at least a portion of the particles are implanted into the hard tissues. The jet device may be of any suitable jet device, such as described for device 100, 200. In one example, the device may be an air jet device or an abrasive air jet device. In another example, the device may be a fluid, e.g., water, jet device.

The jet device can produce any suitable air pressure sufficient to facilitate remineralization of the hard tissues without any severe damage. The air pressure would vary depending up on the nature of the particles 101, the nature of the hard tissue 109a,b,c, the nature of the location of the remineralization, and the nature of the jet device 100, 200. In one example, the device produces air pressure from about 10 psi to about 200 psi. In another example, the air pressure can be in the range form about 1 atmosphere to about 10 atmospheres. A person skilled in the art would appreciate that air pressure can be adjusted depending up on the application.

The nozzle 106, 214 of the jet device may be angled in relation to the device component to which the nozzle is attached. The component can be a handpiece. The nozzle's angle in relation to the component to which it is attached may vary. For example, the nozzle can be attached to the component at any angle ranging from about 30 degrees to about 180 degrees.

The nozzle may have any suitable inner diameter that would facilitate implantation of the particles into hard tissues by delivering the particles with sufficient velocity. In one example, the inner diameter of the nozzle can be from about 0.005 mm to about and 5 mm. In another example, the inner diameter of the nozzle can be from about 0.2 mm to about 2 mm.

The nozzle may be held at any angle in relation to the hard tissues. For example, the nozzle is held at an angle of about 0 degrees to about 180 degrees, for example, but not limited to about 40 degrees to about 120 degrees, in relation to the hard tissues. The nozzle may be held at any suitable distance from the hard tissues such that the implantation of the particles into hard tissues is optimized. In one example, the nozzle of the device is held at a distance of about 0.1 mm to about 25 mm, preferably 1 mm to about 25 mm, from the hard tissues. The nozzle may deposit the particles onto the hard tissues at any suitable flow rate or velocity. In one example, the particles are deposited onto the hard tissues at a gravimetric flow rate ranging from about 0.1 g/s to 5.0 g/s.

The method 300 provides for the remineralization of hard tissues. Remineralization involves delivery of the particles to the hard tissues, for example to the demineralized zones of the hard tissues, such that the particles remain within this region long enough for solid mineral ions to be precipitated out of the particles and then incorporated within the scaffold of the hard tissue. In one example, particle delivery may have a duration about 1 second to about 5 minutes. The particle delivery onto the hard tissues may be either continuous or in intermittent pulses. In some examples, the remineralization can take place over a prolonged time after the delivery of the particles.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A method for remineralizing a hard tissue in a subject, comprising:
directing a pressurized stream of particles comprising one or both of calcium or phosphorus onto a site of interest on the hard tissue thereby implanting at least a portion of the particles therewithin, said implanted particles effective to deliver one or both of calcium or phosphorus ions to the hard tissue thereby reminerlizing the hard tissue in the subject.

2. The method of claim 1, further comprising:
positioning a nozzle operably attached to a jet delivery device adapted to deliver the particles therethrough proximately to the site of interest on the hard tissue.

3. The method of claim 2, wherein nozzle has a diameter of about 0.005 mm and to about 10 mm.

4. The method of claim 2, wherein the nozzle is positioned about 1 mm to about 25 mm from the hard tissue.

5. The method of claim 2, wherein the nozzle is angled or is positioned at an angle from the hard tissue.

6. The method of claim 5, wherein the nozzle is positioned at an angle of about 0 degrees to about 180 degrees relative to the hard tissues.

7. The method of claim 2, wherein the device is an air jet device or an aqueous jet device.

8. The method of claim 1, wherein a component mass of a particle is about 2 percent to about 30 percent phosphorus and about 25 percent to about 60 percent calcium.

9. The method of claim 1, wherein the particles are apatite or hydroxyapatite.

10. The method of claim 1, wherein the particles further comprise one or more abrasives.

11. The method of claim 10, wherein the abrasives include aluminum oxide, crushed glass, glass beads, plastic media, silicon carbide, sodium bicarbonate, or walnut shell.

12. The method of claim 1, wherein particle size is about 0.001 μm to about 1000 μm.

13. The method of claim 1, wherein the particles have an absolute hardness of about 0.1 to about 2000.

14. The method of claim 1, wherein the particles are deposited onto the hard tissue at a flow rate of about 0.1 g/s to about 5.0 g/s.

15. The method of claim 1, wherein the pressurized stream is an air stream or an aqueous stream.

16. The method of claim 1, wherein the hard tissue is enamel, dentin or bone.

17. The method of claim 1, wherein the site of interest is a bacterial lesion or a demineralized area of the hard tissue.

18. A method for remineralizing a tooth in a subject, comprising:
delivering via a jet device a plurality of apatite particles to a site of demineralization on the tooth at a flow rate whereby at least a portion of the apatite particles are implanted therein; said implanted apatite particles effective to deliver both calcium and phosphous ions to the demineralized site thereby remineralizing the tooth in the subject.

19. The method of claim 18, wherein a component mass of a particle is about 2 percent to about 30 percent phosphorus and about 25 percent to about 60 percent calcium.

20. The method of claim 18, wherein the apatite particle size is about 1 μm to about 1000 μm.

21. The method of claim 18, wherein the apatite is a hydroxyapatite with a particle size of about 1 μm to about 100 μm.

22. The method of claim 18, wherein the apatite particles further comprise one or more abrasives including aluminum oxide, crushed glass, glass beads, plastic media, silicon carbide, sodium bicarbonate, or walnut shell.

23. The method of claim 18, wherein the particles have an absolute hardness of about 0.1 to about 2000.

24. The method of claim 23, wherein the particles have a hardness of about 1 Mohs to about 20 Mohs.

25. The method of claim 18, wherein the flow rate is about 0.1 g/s to about 5.0 g/s.

26. The method of claim 18, wherein the apatite particles are delivered at an angle of about 0 degrees to about 180 degrees relative to the tooth.

27. The method of claim 18, wherein the site of demineralization is on one or both of the enamel or the dentin of the tooth.

28. A method for remineralizing a bone in a subject, comprising:
delivering via a jet device a plurality of particles comprising one or both of calcium or phosphorus to a site of demineralization on the bone at a flow rate whereby at least a portion of the particles are implanted therein; said implanted particles effective to deliver one or both of calcium or phosphous ions to the demineralized site thereby remineralizing the bone in the subject.

29. The method of claim 28, wherein a component mass of a particle is about 2 percent to about 30 percent phosphorus and about 25 percent to about 60 percent calcium.

30. The method of claim 28, wherein the particles are apatite or hydroxyapatite.

31. The method of claim 28, wherein the particles further comprise one or more abrasives including aluminum oxide, crushed glass, glass beads, plastic media, silicon carbide, sodium bicarbonate, or walnut shell.

32. The method of claim 28, wherein particle size is about 0.001 μm to about 1000 μm.

33. The method of claim 28, wherein the particles have an absolute hardness of about 0.1 to about 1500.

34. The method of claim 28, wherein the particles are delivered an angle of about 0 degrees to about 180 degrees relative to the bone.

35. The method of claim 28, wherein the particles are deposited onto the bone at a flow rate of about 0.1 g/s to about 5.0 g/s.
